SEQUENCE LISTING (22 sequences)

SEQ ID NO: 1 - protein, 781 aa, Human parvovirus B19
MSKESGKWWESDDEFAKAVYQQFVEFYEKVTGTDLELIQILKDHYNISLDNPLENPSSLFDLVARIKNNLKNSPDLYSHHFQSHGQLSDHPHALSSSSSHAEPRGEDAVLSSEDLHKPGQVSVQLPGTNYVGPGNELQAGPPQSAVDSAARIHDFRYSQLAKLGINPYTHWTVADEELLKNIKNETGFQAQVVKDYFTLKGAAAPVAHFQGSLPEVPAYNASEKYPSMTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 2 - protein, 554 aa, Human parvovirus B19
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 3 - DNA, 397 bp, Human parvovirus B19
gcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggtcagttttgtaacggttaaaatgggcggagcgtaggcggggactacagtatatatagcacagcactgccgcagctctttctttctgggctgctttttcctggactttcttgctgttttttgtgagctaactaac

SEQ ID NO: 4 - DNA, 15 bp, Artificial Sequence
  Description of Artificial Sequence Synthetic oligonucleotide
ggatcccgacgatcc

SEQ ID NO: 5 - DNA, 19 bp, Artificial Sequence
  Description of Artificial Sequence Synthetic primer
cctgggcaagttagcgtac

SEQ ID NO: 6 - DNA, 22 bp, Artificial Sequence
  Description of Artificial Sequence Synthetic primer
atgaatccttgcagcactgtca

SEQ ID NO: 7 - DNA, 15 bp, Artificial Sequence
  Description of Artificial Sequence Synthetic probe
tgttgggcctggcaa

SEQ ID NO: 8 - DNA, 20 bp, Artificial Sequence
  Description of Artificial Sequence Synthetic primer
cccgtcaaatccatgtggtc

SEQ ID NO: 9 - DNA, 20 bp, Artificial Sequence
  Description of Artificial Sequence Synthetic primer
agtggtgctctgggtcgtag

SEQ ID NO: 10 - DNA, 20 bp, Artificial Sequence
  Description of Artificial Sequence Synthetic probe
cttctctgccaacagcgtca

SEQ ID NO: 11 - DNA, 19 bp, Artificial Sequence
  Description of Artificial Sequence Synthetic primer
ggcacccagcacaatgaag

SEQ ID NO: 12 - DNA, 20 bp, Artificial Sequence
  Description of Artificial Sequence Synthetic primer
gccgatccacacggagtact

SEQ ID NO: 13 - DNA, 26 bp, Artificial Sequence
  Description of Artificial Sequence Synthetic probe
tcaagatcattgctcctcctgagcgc

SEQ ID NO: 14 - DNA, 2347 bp, Artificial Sequence
  Description of Artificial Sequence Synthetic polynucleotide
atgagtaaggaatctggcaaatggtgggagtcagacgatgaatttgcgaaggccgtgtaccagcaattcgtggagttctatgaaaaggtaactgggacagatctcgaattgatacaaattctaaaggaccattacaatattagccttgataaccctttagagaacccttcgagcctttttgatctcgtcgctcgcatcaagaataacctgaagaactcccccgacttgtactcccaccattttcagtcacatgggcagctgagcgatcacccacacgccctttctagcagttcttcccacgcagagccaaggggagaggacgcggtactgagttcagaagacctgcacaaacccggacaggtctccgttcagctgccgggcacaaattacgttggccccggcaatgagctccaagcaggcccacctcagtcagccgtggactccgccgctagaatccatgacttccggtacagccagctggctaaattaggtatcaacccttatactcactggaccgttgcagacgaggaactgttgaaaaatattaaaaatgagaccggattccaggcccaggtggtcaaggattacttcacgctgaagggtgctgccgcaccggtggcccattttcaggggagtctccccgaggtgcccgcctataacgcttctgaaaaatatccaagcatgaccagcgttaactctgccgaggccagcacaggcgctggtgggggaggcagcaaccccgtcaaatccatgtggtccgagggcgccaccttctctgccwmyagmgtcacctgcacattcagccggcagtttctgattccctacgacccagagcaccactataaggtgtttagcccagctgccagctcctgccacaacgcaagcggcaaggaggccaaggtgtgtactatcagccccatcatgggttattccactccctggcgctacctagacttcaacgctctcaacctgtttttctcccccctcgagttccagcacctgatcgaaaactacgggagcatcgcccccgacgctctgaccgtcaccataagcgagatcgccgtaaaggatgttactgataagacaggcggtggcgtgcaagtcaccgattccacaacaggccgcctctgtatgctcgtagaccacgagtacaagtacccctatgtcctgggccagggtcaggacaccctcgctccagagctgcctatctgggtgtactttcccccccaatacgcctacctaactgtcggggacgtcaacacacaaggcatcagcggcgactctaaaaaactcgcctccgaggagtccgcattttacgtgctcgagcacagcagctttcaactgctcggcaccgggggaaccgccaccatgtcttacaagttccctcccgtgccccccgaaaatctcgaagggtgcagccagcatttctacgagatgtaacaaccccctgtacggctccaggctcggtgtgcccgataccctcggcggtgatcccaagtttaggagcctcacccacgaggaccatgcaatccagccacaaaacttcatgccagggcccctggttaattctgtcagcaccaaggagggcgattcctccaacaccggcgcagggaaagcactcaccgggctgtccaccggcacttctcaaaacacccgcataagcctcaggccaggaccagttagccaaccctaccaccactgggacacagacaaatacgtgaccgggatcaatgccatcagccacgggcaaaccacctacggcaacgcagaagacaaggaataccagcagggcgtcggtaggttcccaaacgaaaaggagcaacttaaacagctgcagggcctcaatatgcacacctacttccccaacaagggcacccagcaatatacagaccagatcgaaaggcccctcatggttggttccgtgtggaaccgaagagcactgcactacgagtcccaactgtggtccaagatccccaacctagacgacagcttcaagacacaatttgccgcactaggcgggtggggcctgcaccagcccccacctcaaatcttcctcaagatcctgccccagtccgggcccatcggcgggataaagagcatgggcatcaccaccctggtccagtacgccgtcggcatcatgactgtgaccatgaccttcaagctcggcccacggaaggcaaccggtcggtggaaccctcaacccggcgtgtatccaccccacgccgcaggccatctgccatacgtcctctacgatccaaccgctacagacgccaagcagcatcacaggcacggctacgaaaagcccgaagagctctggaccgctaaaagcagggttcatccactgtaa

SEQ ID NO: 15 - DNA, 2345 bp, Human parvovirus B19
atgagtaaagaaagtggcaaatggtgggaaagtgatgatgaatttgctaaagctgtgtatcagcaatttgtggaattttatgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactggctaagttgggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttctagacagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtatactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaagcctgaagaattgtggacagccaaaagccgtgtgcacccattgtaa

SEQ ID NO: 16 - DNA, 5596 bp, Human parvovirus B19
ccaaatcagatgccgccggtcgccgccggtaggcgggacttccggtacaagatggcggacaattacgtcatttcctgtgacgtcatttcctgtgacgtcacttccggtgggcgggacttccggaattagggttggctctgggccagcttgcttggggttgccttgacactaagacaagcggcgcgccgcttgatcttagtggcacgtcaaccccaagcgctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttggtgtcttcttttaaattttagcgggcttttttcccgccttatgcaaatgggcagccattttaagtgttttactataattttattggtcagttttgtaacggttaaaatgggcggagcgtaggcggggactacagtatatatagcacagcactgccgcagctctttctttctgggctgctttttcctggactttcttgctgttttttgtgagctaactaacaggtatttatactacttgttaatatactaacatggagctatttagaggggtgcttcaagtttcttctaatgttctggactgtgctaacgataactggtggtgctctttactagatttagacacttctgactgggaaccactaactcatactaacagactaatggcaatatacttaagcagtgtggcttctaagcttgaccttaccggggggccactagcagggtgcttgtacttttttcaagcagaatgtaacaaatttgaagaaggctatcatattcatgtggttattggggggccagggttaaaccccagaaacctcacagtgtgtgtagaggggttatttaataatgtactttatcactttgtaactgaaaatgtgaagctaaaatttttgccaggaatgactacaaaaggcaaatactttagagatggagagcagtttatagaaaactatttaatgaaaaaaatacctttaaatgttgtatggtgtgttactaatattgatggatatatagatacctgtatttctgctacttttagaaggggagcttgccatgccaagaaaccccgcattaccacagccataaatgatactagtagcgatgctggggagtctagcggcacaggggcagaggttgtgccatttaatgggaagggaactaaggctagcataaagtttcaaactatggtaaactggttgtgtgaaaacagagtgtttacagaggataagtggaaactagttgactttaaccagtacactttactaagcagtagtcacagtggaagttttcaaattcaaagtgcactaaaactagcaatttataaagcaactaatttagtgcctactagcacatttttattgcatacagactttgagcaggttatgtgtattaaagacaataaaattgttaaattgttactttgtcaaaactatgaccccctattggtggggcagcatgtgttaaagtggattgataaaaaatgtggcaagaaaaatacactgtggttttatgggccgccaagtacaggaaaaacaaacttggcaatggccattgctaaaagtgttccagtatatggcatggttaactggaataatgaaaactttccatttaatgatgtagcaggaaaaagcttggtggtctgggatgaaggtattattaagtctacaattgtagaagctgcaaaagccattttaggcgggcaacccaccagggtagatcaaaaaatgcgtggaagtgtagctgtgcctggagtacctgtggttataaccagcaatggtgacattacttttgttgtaagcgggaacactacaacaactgtacatgctaaagccttaaaagagcgcatggtaaagttaaactttactgtaagatgcagccctgacatggggttactaacagaggctgatgtacaacagtggcttacatggtgtaatgcacaaagctgggaccactatgaaaactgggcaataaactacacttttgatttccctggaattaatgcagatgccctccacccagacctccaaaccaccccaattgtcacagacaccagtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagcccagtttcctccgaagttgtagctgcatcgtgggaagaagccttctacacacctttggcagaccagtttcgtgaactgttagttggggttgattatgtgtgggacggtgtaaggggtttacctgtgtgttgtgtgcaacatattaacaatagtgggggaggcttgggactttgtccccattgcattaatgtaggggcttggtataatggatggaaatttcgagaatttaccccagatttggtgcgatgtagctgccatgtgggagcttctaatcccttttctgtgctaacctgcaaaaaatgtgcttacctgtctggattgcaaagctttgtagattatgagtaaagaaagtggcaaatggtgggaaagtgatgatgaatttgctaaagctgtgtatcagcaatttgtggaattttatgaaaaggttactggaacagacttagagcttattcaaatattaaaagatcattataatatttctttagataatcccctagaaaacccatcctctctgtttgacttagttgctcgcattaaaaataaccttaaaaattctccagacttatatagtcatcattttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagccaactggctaagttgggaataaatccatatactcattggactgtagcagatgaagagcttttaaaaaatataaaaaatgaaactgggtttcaagcacaagtagtaaaagactactttactttaaaaggtgcagctgcccctgtggcccattttcaaggaagtttgccggaagttcccgcttacaacgcctcagaaaaatacccaagcatgacttcagttaattctgcagaagccagcactggtgcaggaggggggggcagtaatcctgtcaaaagcatgtggagtgagggggccacttttagtgccaactctgtgacttgtacattttctagacagtttttaattccatatgacccagagcaccattataaggtgttttctcccgcagcaagtagctgccacaatgccagtggaaaggaggcaaaggtttgcaccattagtcccataatgggatactcaaccccatggagatatttagattttaatgctttaaacttatttttttcacctttagagtttcagcacttaattgaaaattatggaagtatagctcctgatgctttaactgtaaccatatcagaaattgctgttaaggatgttacagacaaaactggagggggggtgcaggttactgacagcactacagggcgcctatgcatgttagtagaccatgaatacaagtacccatatgtgttagggcaaggtcaagatactttagccccagaacttcctatttgggtatactttccccctcaatatgcttacttaacagtaggagatgttaacacacaaggaatttctggagacagcaaaaaattagcaagtgaagaatcagcattttatgttttggaacacagttcttttcagcttttaggtacaggaggtacagcaactatgtcttataagtttcctccagtgcccccagaaaatttagagggctgcagtcaacacttttatgagatgtacaatcccttatacggatcccgcttaggggttcctgacacattaggaggtgacccaaaatttagatctttaacacatgaagaccatgcaattcagccccaaaacttcatgccagggccactagtaaactcagtgtctacaaaggagggagacagctctaatactggagctgggaaagccttaacaggccttagcacaggtacctctcaaaacactagaatatccttacgcccggggccagtgtctcagccgtaccaccactgggacacagataaatatgtcacaggaataaatgctatttctcatggtcagaccacttatggtaacgctgaagacaaagagtatcagcaaggagtgggtagatttccaaatgaaaaagaacagctaaaacagttacagggtttaaacatgcacacctactttcccaataaaggaacccagcaatatacagatcaaattgagcgccccctaatggtgggttctgtatggaacagaagagcccttcactatgaaagccagctgtggagtaaaattccaaatttagatgacagttttaaaactcagtttgcagccttaggaggatggggtttgcatcagccacctcctcaaatatttttaaaaatattaccacaaagtgggccaattggaggtattaaatcaatgggaattactaccttagttcagtatgccgtgggaattatgacagtaaccatgacatttaaattggggccccgtaaagctacgggacggtggaatcctcaacctggagtatatcccccgcacgcagcaggtcatttaccatatgtactatatgaccctacagctacagatgcaaaacaacaccacagacatggatatgaaaagcctgaagaattgtggacagccaaaagccgtgtgcacccattgtaaacactccccaccgtgccctcagccaggatgcgtaactaaacgcccaccagtaccacccagactgtacctgccccctcctatacctataagacagcctaacacaaaagatatagacaatgtagaatttaagtatttaaccagatatgaacaacatgttattagaatgttaagattgtgtaatatgtatcaaaatttagaaaaataaacgtttgttgtggttaaaaaattatgttgttgcgctttaaaaatttaaaagaagacaccaaatcagatgccgccggtcgccgccggtaggcgggacttccggtacaagatggcggacaattacgtcatttcctgtgacgtcatttcctgtgacgtcacttccggtgggcggaacttccggaattagggttggctctgggccagcgcttggggttgacgtgccactaagatcaagcggcgcgccgcttgtcttagtgtcaaggcaaccccaagcaagctggcccagagccaaccctaattccggaagtcccgcccaccggaagtgacgtcacaggaaatgacgtcacaggaaatgacgtaattgtccgccatcttgtaccggaagtcccgcctaccggcggcgaccggcggcatctgatttgg

SEQ ID NO: 17 - protein, 671 aa, Human parvovirus B19
MELFRGVLQVSSNVLDCANDNWWCSLLDLDTSDWEPLTHTNRLMAIYLSSVASKLDLTGGPLAGCLYFFQAECNKFEEGYHIHVVIGGPGLNPRNLTVCVEGLFNNVLYHFVTENVKLKFLPGMTTKGKYFRDGEQFIENYLMKKIPLNVVWCVTNIDGYIDTCISATFRRGACHAKKPRITTAINDTSSDAGESSGTGAEVVPFNGKGTKASIKFQTMVNWLCENRVFTEDKWKLVDFNQYTLLSSSHSGSFQIQSALKLAIYKATNLVPTSTFLLHTDFEQVMCIKDNKIVKLLLCQNYDPLLVGQHVLKWIDKKCGKKNTLWFYGPPSTGKTNLAMAIAKSVPVYGMVNWNNENFPFNDVAGKSLVVWDEGIIKSTIVEAAKAILGGQPTRVDQKMRGSVAVPGVPVVITSNGDITFVVSGNTTTTVHAKALKERMVKLNFTVRCSPDMGLLTEADVQQWLTWCNAQSWDHYENWAINYTFDFPGINADALHPDLQTTPIVTDTSISSSGGESSEELSESSFFNLITPGAWNTETPRSSTPIPGTSSGESFVGSPVSSEVVAASWEEAFYTPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 18 - protein, 74 aa, Human parvovirus B19
MQMPSTQTSKPPQLSQTPVSAAVVVKALKNSVKAAFLTSSPQAPGTLKPRALVRPSPGPVQENHLSEAQFPPKL

SEQ ID NO: 19 - protein, 81 aa, Human parvovirus B19
MDSYLTTPMPYHPVAVMQNLEEKMQYYLVKTYTSLGKLAYNYPVLTMLGLAMSYKLGPRKVLLTVLQGFMTLGIANWLSWE

SEQ ID NO: 20 - protein, 94 aa, Human parvovirus B19
MQNNTTDMDMKSLKNCGQPKAVCTHCKHSPPCPQPGCVTKRPPVPPRLYLPPPIPIRQPNTKDIDNVEFKYLTRYEQHVIRMLRLCNMYQNLEK

SEQ ID NO: 21 - DNA, 2346 bp, Artificial Sequence
  Description of Artificial Sequence Synthetic polynucleotide
atgagtaaggaatctggcaaatggtgggagtcagacgatgaatttgcgaaggccgtgtaccagcaattcgtggagttctatgaaaaggtaactgggacagatctcgaattgatacaaattctaaaggaccattacaatattagccttgataaccctttagagaacccttcgagcctttttgatctcgtcgctcgcatcaagaataacctgaagaactcccccgacttgtactcccaccattttcagtcacatgggcagctgagcgatcacccacacgccctttctagcagttcttcccacgcagagccaaggggagaggacgcggtactgagttcagaagacctgcacaaacccggacaggtctccgttcagctgccgggcacaaattacgttggccccggcaatgagctccaagcaggcccacctcagtcagccgtggactccgccgctagaatccatgacttccggtacagccagctggctaaattaggtatcaacccttatactcactggaccgttgcagacgaggaactgttgaaaaatattaaaaatgagaccggattccaggcccaggtggtcaaggattacttcacgctgaagggtgctgccgcaccggtggcccattttcaggggagtctccccgaggtgcccgcctataacgcttctgaaaaatatccaagcatgaccagcgttaactctgccgaggccagcacaggcgctggtgggggaggcagcaaccccgtcaaatccatgtggtccgagggcgccaccttctctgccwmyagmgtcacctgcacattcagccggcagtttctgattccctacgacccagagcaccactataaggtgtttagcccagctgccagctcctgccacaacgcaagcggcaaggaggccaaggtgtgtactatcagccccatcatgggttattccactccctggcgctacctagacttcaacgctctcaacctgtttttctcccccctcgagttccagcacctgatcgaaaactacgggagcatcgcccccgactctgaccgtcaccataagcgagatcgccgtaaaggatgttactgataagacaggcggtggcgtgcaagtcaccgattccacaacaggccgcctctgtatgctcgtagaccacgagtacaagtacccctatatgtcctgggccagggtcaggacaccctcgctccagagctgcctatctgggtgtactttcccccccaatacgcctacctaactgtcggggacgtcaacacacaaggcatcagcggcgactctaaaaaactcgcctccgaggagtccgcattttacgtgctcgagcacagcagctttcaactgctcggcaccgggggaaccgccaccatgtcttacaagttccctcccgtgccccccgaaaatctcgaagggtgcagccagcatttctacgagatgtacaaccccctgtacggctccaggctcggtgtgcccgataccctcggcggtgatcccaagtttaggagcctcacccacgaggaccatgcaatccagccacaaaacttcatgccagggcccctggttaattctgtcagcaccaaggagggcgattcctccaacaccggcgcagggaaagcactcaccgggctgtccaccggcacttctcaaaacacccgcataagcctcaggccaggaccagttagccaaccctaccaccactgggacacagacaaatacgtgaccgggatcaatgccatcagccacgggcaaaccacctacggcaacgcagaagacaaggaataccagcagggcgtcggtaggttcccaaacgaaaaggagcaacttaaacagctgcagggcctcaatatgcacacctacttccccaacaagggcacccagcaatatacagaccagatcgaaaggcccctcatggttggttccgtgtggaaccgaagagcactgcactacgagtcccaactgtggtccaagatccccaacctagacgacagcttcaagacacaatttgccgcactaggcgggtggggcctgcaccagcccccacctcaaatcttcctcaagatcctgccccagtccgggcccatcggcgggataaagagcatgggcatcaccaccctggtccagtacgccgtcggcatcatgactgtgaccatgaccttcaagctcggcccacggaaggcaaccggtcggtggaaccctcaacccggcgtgtatccaccccacgccgcaggccatctgccatacgtcctctacgatccaaccgctacagacgccaagcagcatcacaggcacggctacgaaaagcccgaagagctctggaccgctaaaagcagggttcatccactgtaa

SEQ ID NO: 22 - protein, 781 aa, Artificial Sequence
  Description of Artificial Sequence Synthetic polypeptide
MSKESGKWWESDDEFAKAVYQQFVEFYEKVTGTDLELIQILKDHYNISLDNPLENPSSLFDLVARIKNNLKNSPDLYSHHFQSHGQLSDHPHALSSSSSHAEPRGEDAVLSSEDLHKPGQVSVQLPGTNYVGPGNELQAGPPQSAVDSAARIHDFRYSQLAKLGINPYTHWTVADEELLKNIKNETGFQAQVVKDYFTLKGAAAPVAHFQGSLPEVPAYNASEKYPSMTSVNSAEASTGAGGGGSNPVKSMWSEGATFSAXXVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

(19) United States
(12) Patent Application Publication
(10) Pub. No.: US 2013/0052226 A1
(43) Pub. Date: Feb. 28, 2013

(54) COMPOSITIONS AND METHODS FOR PREVENTING OR TREATING A HUMAN PARVOVIRUS INFECTION

(71) Applicants: Ning Zhi, Potomac, MD (US); Neal S. Young, Washington, DC (US); Sachiko Kajigaya, Rockville, MD (US)

(72) Inventors: Ning Zhi, Potomac, MD (US); Neal S. Young, Washington, DC (US); Sachiko Kajigaya, Rockville, MD (US)

(73) Assignee: The Government of the USA, as represented by the Secretary, Department of Health & Human Services, Bethesda, MD (US)

(21) Appl. No.: 13/578,577

(22) PCT Filed: Feb. 9, 2011
(86) PCT No.: PCT/US11/24199

§ 371 Date: Nov. 13, 2012

Related U.S. Application Data
(60) Provisional application No. 61/337,983, filed on Feb. 12, 2010.

(51) Int. Cl. A61K 39/23; A61P 37/04; C12N 5/10; C12N 15/85; C12P 21/02; C12N 15/35; A61P 31/20

(52) U.S. Cl. 424/233.1; 536/23.72; 435/320.1; 435/69.1; 435/369; 435/365; 435/367; 435/372

(57) ABSTRACT

The invention provides a codon-optimized parvovirus polynucleotide composition and methods of expressing this polynucleotide in a variety of mammalian cells, including non-erythroid progenitor cells, to produce immunogenic compositions.

### CROSS-REFERENCE TO RELATED APPLICATION

This application claims the benefit of U.S. Provisional Application No. 61/337,983, filed Feb. 12, 2010, the contents of which is incorporated herein by reference in its entirety.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The government has certain rights in the invention.

### BACKGROUND OF THE INVENTION

Parvovirus B19 (B19V) is a human pathogenic parvovirus. The virus has an extreme tropism for human erythroid progenitors, targeting human erythroid (red cell) progenitors found in blood, bone marrow, and fetal liver. Replication of B19V in continuous cell lines is also restricted. Few semi-permissive cell lines have been described and those few that exist fail to express virus in amounts suitable for vaccine production. Cellular receptors and cellular factors that function in viral DNA replication and RNA maturation are thought to be related to the restricted permissiveness for B19V propagation. Nevertheless, the preferential propagation of B19V in erythroid progenitors is not fully understood. Antiviral drugs are not available for the treatment of parvovirus B19V infection, and no vaccines for the virus are currently approved. Therefore, improved compositions and methods for developing a prophylactic or therapeutic vaccine are urgently required.

### SUMMARY OF THE INVENTION

As described below, the present invention provides compositions for producing a parvovirus immunogenic composition and methods of using such compositions for the treatment or prevention of a parvovirus infection.

In one aspect, the invention generally provides a nucleic acid molecule encoding a parvovirus structural protein or fragment thereof, where at least about 50-100% of the nucleic acid molecule's codons are optimized for expression in a nonpermissive mammalian cell. In one embodiment, the nonpermissive mammalian cell is a non-erythroid progenitor cell. In another embodiment, the nonpermissive mammalian cell that is any one or more of 293T cells, COS cells, HeLa cells and UT7/Epo-S1 cells.

In another aspect, the invention provides a nucleic acid molecule encoding a parvovirus B19 (B19V) structural protein or fragment thereof, where the codons of the nucleic acid molecule are optimized for expression in a mammalian non-erythroid lineage cell. In one embodiment, the non-erythroid lineage cell is any one or more of 293T cells, COS cells, HeLa cells and UT7/Epo-S1 cells. In another embodiment, the structural protein is a capsid protein. In another embodiment, the capsid protein is VP1 or VP2. In another embodiment, VP1 and VP2 have at least about 85% amino acid identity to the sequence provided at NCBI Accession No. AAQ91879.1 and AAQ91880.1, respectively. In still another embodiment, the B19V structural protein is a VP1 protein containing an altered PLA2 motif (e.g., a PLA2 deletion, a H153A mutation, a D175A mutation, and a P133R mutation) that lacks or has reduced inflammatory properties when injected into a subject relative to a wild-type PLA2 motif. In still another embodiment, the parvovirus structural protein is human B19V VP1 or VP2.

In another aspect, the invention provides an expression vector encoding a parvovirus structural protein or fragment thereof, where at least about 50-100% (e.g., 50, 60, 70, 80, 90, 100%) of the nucleic acid molecule's codons are optimized for expression in a nonpermissive mammalian cell or a non-erythroid lineage cell. In one embodiment, the vector comprises a parvovirus promoter. In another embodiment, the promoter is p6. In another embodiment, the vector comprises a parvovirus 3′UTR. In another embodiment, the vector comprises a codon-optimized VP1 and/or VP2 gene. In still another embodiment, the vector is any one or more of pcDNA(p6)-OptVP2, pcDNA(p6)-OptVP2-3′UTR, pcDNA(pCMV)-OptVP2, and pcDNA(pCMV)-OptVP2-3′UTR.

In another aspect, the invention provides an expression vector containing a CMV promoter positioned to control the expression of a first nucleic acid molecule encoding a parvo VP2 polypeptide and a second nucleic acid molecule encoding a parvo VP1 polypeptide, where the second nucleic acid molecule is separated from the first nucleic acid molecule by one or more inverted repeats. In one embodiment, the presence of the inverted repeats is sufficient to reduce the expression of VP1 relative to VP2. In another embodiment, the presence of the inverted repeats generates a VP2:VP1 ratio of 95:5. In another embodiment, at least about 50-100% of the nucleic acid molecule's codons are optimized for expression in a nonpermissive mammalian cell or a non-erythroid lineage cell. In another embodiment, the codon-optimized VP1 or VP2 protein expression is increased in a nonpermissive or non-erythroid lineage cell relative to wild-type VP1 or VP2 expression in said cell. In another embodiment, the vector is a mammalian bicistronic expression vector. In still another embodiment, vector is pIRES. In another embodiment, the vector further contains an inverted-repeat (ITR) sequence immediately upstream of the VP1 gene. In one embodiment, the vector is any one or more of pIRES-Opt-VP2, pIRES-Opt-VP2/VP1, and pIRES-Op-VP2-ITR-VP1. In another embodiment, the expression vector is pIRES-Opt-VP2-ITR-VP1.

In another aspect, the invention provides an expression vector containing an inducible promoter and a nucleic acid molecule of any previous aspect or otherwise delineated herein. In another embodiment, the vector comprises a tetracycline inducible promoter.

In another aspect, the invention provides a mammalian expression vector containing a nucleic acid molecule of any previous aspect or otherwise delineated herein.

In another aspect, the invention provides a cell containing the expression vector of any previous aspect or otherwise delineated herein. In one embodiment, the cell is nonpermissive for expression of a parvo structural protein or is a nonerythroid lineage cell.

In another embodiment, the cell is any one or more of 293T cells, COS cells, HeLa cells and UT7/Epo-S1 cells

In another aspect, the invention provides a method of producing a virus like particle involving introducing into a nonpermissive or non-erythroid mammalian cell an expression vector containing a nucleic acid molecule encoding a parvovirus structural protein or fragment thereof, where at least about 50-100% of the nucleic acid molecule's codons are optimized for expression in a nonpermissive mammalian cell; culturing the cell under conditions to produce the structural proteins and form the VLP; and isolating the VLP.

In another aspect, the invention provides a method of producing a virus like particle involving introducing into a nonpermissive or non-erythroid mammalian cell an expression vector according to any previous aspect; culturing the cell under conditions to produce the structural proteins and form the VLP; and isolating the VLP.

In another aspect, the invention provides an immunogenic composition containing a nucleic acid molecule encoding a parvovirus structural protein or fragment thereof, where at least about 50-100% of the nucleic acid molecule's codons are optimized for expression in a nonpermissive mammalian cell. In one embodiment, the nonpermissive mammalian cell is a non-erythroid progenitor cell.

In another aspect, the invention provides an immunogenic composition containing a nucleic acid molecule encoding a parvovirus B19 (B19V) structural protein or fragment thereof, where the codons of the nucleic acid molecule are optimized for expression in a mammalian non-erythroid lineage cell. In one embodiment, VP1 and VP2 have at least about 85% amino acid identity to the sequence provided at NCBI Accession No. AAQ91879.1 and AAQ91880.1, respectively. In another embodiment, the B19V structural protein is a VP1 protein containing an altered PLA2 motif (e.g., a PLA2 deletion, a H153A mutation, a D175A mutation, and a P133R mutation) that lacks or has reduced inflammatory properties when injected into a subject relative to a wild-type PLA2 motif. In another embodiment, the parvovirus structural protein is human B19V VP1 or VP2.

In another aspect, the invention provides an immunogenic composition containing a combination of an effective amount of the immunogenic composition of a previous aspect and an effective amount of a VLP containing human B19V VP1 and VP2.

In another aspect, the invention provides an immunogenic composition containing an effective amount of a VLP produced according to a method delineated herein and a pharmaceutically acceptable carrier. In one embodiment, the composition further includes a nucleic acid molecule encoding a parvovirus B19 (B19V) structural protein or fragment thereof, where the codons of the nucleic acid molecule are optimized for expression in a mammalian non-erythroid lineage cell.

In another aspect, the invention provides a method for producing an immune response in a subject, the method involving administering to the subject an effective amount of an immunogenic composition of any of claims 32-43, thereby generating an immune response in said subject.

In another aspect, the invention provides a method for producing an immune response in a subject, the method involving administering to the subject an effective amount of an immunogenic composition containing a nucleic acid molecule encoding a parvovirus B19 (B19V) structural protein or fragment thereof, where the codons of the nucleic acid molecule are optimized for expression in a mammalian non-erythroid lineage cell and a VLP containing human B19V VP1 and VP2. In one embodiment, the immune response comprises production of neutralizing antibodies.

In another aspect, the invention provides a method for treating or preventing a parvovirus infection in a subject, the method involving administering to the subject an effective amount of an immunogenic composition of any aspect of the invention delineated herein; and generating an immune response in said subject, where the immune response prevents or treats a parvovirus infection.

In another aspect, the invention provides a kit containing an effective amount of a nucleic acid molecule encoding a parvovirus B19 (B19V) structural protein or fragment thereof, where the codons of the nucleic acid molecule are optimized for expression in a mammalian non-erythroid lineage cell and instructions for the use of said kit in the method of any previous aspect. In one embodiment, the kit is used for in vitro or in vivo expression of a parvovirus B19 structural protein.

In another aspect, the invention provides a kit containing a nucleic acid molecule encoding a parvovirus B19 (B19V) structural protein or fragment thereof, where the codons of the nucleic acid molecule are optimized for expression in a mammalian non-erythroid lineage cell and a VLP containing human B19V VP1 and VP2, and directions for the use of the kit in the method of any previous aspect.

In various embodiments of any previous aspect or any other aspect of the invention delineated herein, the vector is pIRES-Opt-VP2-ITR-VP1. In other embodiments, the VLP comprises parvo VP2 and VP1, where the VP2:VP1 ratio is about 95:5. In still other embodiments, VP1 and VP2 have at least about 85% amino acid identity to the sequence provided at NCBI Accession No. AAQ91879.1 and AAQ91880.1, respectively. In still other embodiments of the above aspects, the B19V structural protein is a VP1 protein containing an altered PLA2 motif (e.g., a PLA2 deletion, a H153A mutation, a D175A mutation, and a P133R mutation) that lacks or has reduced inflammatory properties when injected into a subject relative to a wild-type PLA2 motif. In other embodiments, the parvovirus structural protein is human B19V VP1 or VP2. In still other embodiments, the subject is a human subject.

The invention provides a codon-optimized parvovirus polynucleotide composition and methods of expressing this polynucleotide in a variety of mammalian cells, including non-erythroid progenitor cells, to produce immunogenic compositions. Compositions and articles defined by the invention were isolated or otherwise manufactured in connection with the examples provided below. Other features and advantages of the invention will be apparent from the detailed description, and from the claims.

### DEFINITIONS

By “codon optimized nucleic acid molecule” is meant that the polynucleotide includes certain sequence alterations relative to a wild-type nucleic acid sequence that provides for the detectable production of an encoded polypeptide in a cell type that does not typically permit the detectable production of such polypeptides. Thus, a “codon optimized nucleic acid molecule” is capable of expression in a nonpermissive mammalian cell. An exemplary codon optimized nucleic acid molecule encoding VP1 and VP2 is provided at FIG. 5.

By “non-erythroid progenitor cell” is meant a cell that does not produce erythroid progeny.

By “non-erythroid lineage cell” is meant a cell that is not an erythroid cell, does not produce erythroid progeny, and/or does not belong to a cell lineage capable of generating an erythroid cell type. Exemplary erythroid lineage cells are hematopoietic and endothelial stem cells. Exemplary non-erythroid lineage cells include, but are not limited to, 293T cells, COS cells, HeLa cells and UT7/Epo-S1 cells.

By “parvovirus structural protein” is meant a polypeptide or fragment thereof that contributes to a parvovirus capsid. In one embodiment, a parvovirus structural protein has at least about 85% amino acid sequence identity to a naturally occurring VP1 or VP2 protein and having immunogenic activity in a mammal. In other embodiments, the amino acid sequence identity is at least about 90%, 95%, or more.

By “VP1 polypeptide” is meant a protein having at least about 85% amino acid identity to NCBI Accession No. AAQ91879.1 or a fragment thereof capable of inducing an immune response in a subject. An exemplary VP1 amino acid sequence is provided below:

| | |
|---|---|
| 1 | mskesgkwwe sddefakavy qqfvefyekv tgtdleliqi lkdhynisld nplenpsslf |
| 61 | dlvariknnl knspdlyshh fqshgqlsdh phalsssssh aeprgedavl ssedlhkpgq |
| 121 | vsvqlpgtny vgpgnelqag ppqsavdsaa rihdfrysql aklginpyth wtvadeellk |
| 181 | niknetgfqa qvvkdyftlk gaaapvahfq gslpevpayn asekypsmts vnsaeastga |
| 241 | ggggsnpvks mwsegatfsa nsvtctfsrq flipydpehh ykvfspaass chnasgkeak |
| 301 | vctispimgy stpwryldfn alnlffsple fqhlienygs iapdaltvti seiavkdvtd |
| 361 | ktgggvqvtd sttgrlcmlv dheykypyvl gqgqdtlape lpiwvyfppq yayltvgdvn |
| 421 | tqgisgdskk laseesafyv lehssfqllg tggtatmsyk fppvppenle gcsqhfyemy |
| 481 | nplygsrlgv pdtlggdpkf rslthedhai qpqnfmpgpl vnsvstkegd ssntgagkal |
| 541 | tglstgtsqn trislrpgpv sqpyhhwdtd kyvtginais hgqttygnae dkeyqqgvgr |
| 601 | fpnekeqlkq lqglnmhtyf pnkgtqqytd qierplmvgs vwnrralhye sqlwskipnl |
| 661 | ddsfktqfaa lggwglhqpp pqiflkilpq sgpiggiksm gittlvqyav gimtvtmtfk |
| 721 | lgprkatgrw npqpgvypph aaghlpyvly dptatdakqh hrhgyekpee lwtaksrvhp |
| 781 | l |

By “VP2 polypeptide” is meant a protein having at least about 85% amino acid identity to NCBI Accession No. AAQ91880.1 or a fragment thereof capable of inducing an immune response in a subject. An exemplary VP2 amino acid sequence is provided below:

| | |
|---|---|
| 1 | mtsvnsaeas tgaggggsnp vksmwsegat fsansvtctf srqflipydp ehhykvfspa |
| 61 | asschnasgk eakvctispi mgystpwryl dfnalnlffs plefghlien ygsiapdalt |
| 121 | vtiseiavkd vtdktgggvq vtdsttgrlc mlvdheykyp yvlgqgqdtl apelpiwvyf |
| 181 | ppqyayltvg dvntqgisgd skklaseesa fyvlehssfq llgtggtatm sykfppvppe |
| 241 | nlegcsqhfy emynplygsr lgvpdtlggd pkfrslthed haiqpqnfmp gplvnsystk |
| 301 | egdssntgag kaltglstgt sqntrislrp gpvsqpyhhw dtdkyvtgin aishgqttyg |
| 361 | naedkeyqqg vgrfpnekeq lkqlqglnmh tyfpnkgtqq ytdqierplm vgsvwnrral |
| 421 | hyesqlwski pnlddsfktq faalggwglh qpppqiflki lpqsgpiggi ksmgittivq |
| 481 | yavgimtvtm tfklgprkat grwnpqpgvy pphaaghlpy vlydptatda kqhhrhgyek |
| 541 | peelwtaksr vhpl |

Wild-type VP1 and VP2 coding sequences are included in the human parvovirus B19 complete sequence, isolate C39 from plasma provided at NCBI Accession No. AY386330 (FIG. 6). The sequence of an exemplary codon optimized nucleic acid molecule that encodes VP1 and VP2 polypeptides is provided at FIG. 5.

By “B19V p6 promoter” is meant a regulatory sequence having at least 85% identity to a nucleic acid sequence delineated herein. An exemplary B19V p6 promoter sequence is provided below.

GCTTGATCTTAGTGGCACGTCAACCCCAAGCGCTGGCCCAGAGCCAACCCTAATTCCGGA
AGTCCCGCCCACCGGAAGTGACGTCACAGGAAATGACGTCACAGGAAATGACGTAATTGT
CCGCCATCTTGTACCGGAAGTCCCGCCTACCGGCGGCGACCGGCGGCATCTGATTTGGTG
TCTTCTTTTAAATTTTAGCGGGCTTTTTTCCCGCCTTATGCAAATGGGCAGCCATTTTAA
GTGTTTTACTATAATTTTATTGGTCAGTTTTGTAACGGTTAAAATGGGCGGAGCGTAGGC
GGGGACTACAGTATATATAGCACAGCACTGCCGCAGCTCTTTCTTTCTGGGCTGCTTTTT
CCTGGACTTTCTTGCTGTTTTTTGTGAGCTAACTAAC

By “nonpermissive mammalian cell” is meant a cell that fails to express detectable levels of infectious virus or that expresses only minimal levels of infectious virus.

By “agent” is meant any small molecule chemical compound, antibody, nucleic acid molecule, or polypeptide, or fragments thereof.

As used herein, the term “adjuvant” is meant to refer to a compound that, when used in combination with a specific immunogen in a formulation, will augment, alter or modify the resultant immune response. In certain embodiments, the adjuvant is used in combination with a VLP. Modification of the immune response includes intensification or broadening the specificity of either or both antibody and cellular immune responses. Modification of the immune response can also mean decreasing or suppressing certain antigen-specific immune responses.

As used herein “inducing immunity” is meant to refer to any immune response generated against an antigen. In one embodiment, immunity is mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, block infectious agents from entering cells, block replication of infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates an infection, for example parvovirus infection, or reduces at least one symptom thereof.

By “ameliorate” is meant decrease, suppress, attenuate, diminish, arrest, or stabilize the development or progression of a disease or a symptom thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. In one embodiment, the invention provides codon optimized nucleic acid molecules that encode parvovirus structural proteins at an increased level in a nonpermissive cell type relative to the expression of a corresponding wild-type nucleic acid molecule in such cells.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disorder that damages or interferes with the normal function of a cell, tissue, or organ. Examples of diseases include parvovirus infections, including parvovirus B19 (B19V) infections.

By “effective amount” is meant the amount of an agent required to ameliorate the symptoms of a disease relative to an untreated patient. The effective amount of active compound(s) used to practice the present invention for prevention or treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunogenic composition” is meant a composition comprising a molecule capable of inducing an immune response in a subject. Such an immune response may be a prophylactic or therapeutic immune response.

By “isolated polynucleotide” is meant a nucleic acid molecule (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “structural protein” is meant a polypeptide that contributes to a viral capsid or envelope. In one embodiment the structural protein is parvovirus VP1 or VP2. In a related embodiment, the structural protein is a parvovirus VP1 protein containing an altered PLA2 motif (e.g., a PLA2 deletion, a H153A mutation, a D175A mutation, and a P133R mutation) that lacks or has reduced inflammatory properties when injected into a subject relative to a wild-type PLA2 motif.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vaccine” refers to a formulation which contains VLPs which is in a form that is capable of being administered to a vertebrate and which induces a protective immune response sufficient to induce immunity to prevent and/or ameliorate an infection and/or to reduce at least one symptom of an infection. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, the term “virus-like particle” (VLP) refers to a structure that in at least one attribute resembles a virus but which has not been demonstrated to be infectious. Virus-like particles in accordance with the invention do not carry genetic information encoding for the proteins of the virus-like particles. In general, virus-like particles lack a viral genome and, therefore, are noninfectious. In addition, virus-like particles can often be produced in large quantities by heterologous expression and can be easily purified.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A and 1B are immunoblots showing the cell type-specific expression of a B19 capsid gene. FIG. 1A is an immunoblot analysis of the production of B19V proteins in non-permissive and semi-permissive cell lines. FIG. 1B is an immunoblot analysis of B19V capsid protein production in CD34⁺ hematopoietic stem cells (HSCs) and CD36⁺ endothelial progenitor cells (EPCs). CD34⁺ HSCs, CD36⁺ EPCs, and UT7/Epo-S1 cells were transfected using the AMAXA Cell Line Nucleofector™ kit R and Hela and 293T cells were transfected using Lipofectamine 2000. Whole cell lysates were prepared at 48 hours post-transfection (hpt), resolved on 4-20% SDS-PAGE and subjected to immunoblot analysis with antibodies specific for Flag-tag. The numbers on the left indicate the molecular masses in kilodaltons based on the broad-range prestained standards (Bio-Rad). Bands were visualized using a SuperSignal chemiluminescent reagent (Pierce) and exposure to X-ray film.

FIGS. 2A and 2B show that codon usage restricts B19V capsid gene expression. FIG. 2A provides schematic diagrams illustrating plasmid construction. FIG. 2B is an immunoblot analysis of B19V capsid protein production in different types of cell lines and primary CD34⁺ HSCs and CD36⁺ EPCs. HeLa and 293T cells were transfected using Lipofectamine 2000. CD34⁺ HSCs, CD36⁺EPCs and UT7/Epo-S1 cells were transfected using the AMAXA Cell Line Nucleofector™ kit R as described elsewhere (Komatsu et al., 1993. Blood 82:456-464). Whole cell lysates were prepared at 48 hpt, resolved on 4-20% SDS-PAGE and subjected to immunoblot analysis with antibodies specific for Flag-tag. The numbers on the left indicate the molecular masses in kilodaltons based on the broad-range prestained standards (Bio-Rad). Bands were visualized by using SuperSignal chemiluminescent reagent (Pierce) and exposure to X-ray film.

FIG. 3 includes two graphs showing that codon optimization has no impact on transcriptional efficiency of the B19V capsid gene. The abundance of wild-type and codon-optimized VP2 transcripts was quantitated by real-time reverse transcription-PCR (RT-PCR). Total RNA was extracted from the cells at 24 hpt and converted to cDNA by using random primers. Real-time RT-PCR was performed using 5 μl of the resulting cDNA, which was amplified as a multiplex with specific probes for wild-type or codon-optimized VP2, and β-actin as an internal control. Quantitations are given as the numbers of transcripts per microliter of RNA extracts, which is normalized with the numbers of transcripts of β-actin. Error bars indicate standard deviations.

FIGS. 4A-4D show the production of B19V virus-like particles (VLP) using a bicistronic vector in non-permissive cells. FIG. 4A provides a chematic diagram that illustrates plasmid construction. FIG. 4B shows an immunoblot analysis of B19V capsid protein production in 293T cells. Cell lysates were prepared using M-PRE Mammalian Protein Extraction Reagent (Pierce) supplemented with Complete Protease Inhibitor Cocktail (Roche). Whole cell extract was subjected to SDS-PAGE and the separated proteins were transferred to nitrocellulose membrane. Antigens were detected by incubation of the membrane with MAb 8293 (Chemicon), followed by incubation with horseradish peroxidase-conjugated anti-mouse. Bands were visualized with enhanced chemiluminescence by incubating the membrane with SuperSignal chemiluminescent reagent (Pierce) and exposing it to X-ray film. The densities of detected bands were analyzed with a PhosphorImager (Molecular Dynamics). FIG. 4C shows a micrograph of cells immunostained with anti-Flag (NS1) antibody and then FITC-conjugated secondary antibody (green). After counterstaining nuclei with DAPI (blue), cells were visualized by confocal microscopy. FIG. 4D includes an electron micrograph (EM) of viral particles. Cells were lysed and clarified by low speed centrifugation. Clarified lysate was layered over 40% sucrose and processed by ultracentrifugation. The pellet was resuspended and analyzed by EM negative stain.

FIG. 5 provides a comparison of wild-type and codon-optimized B19V caspsid genes. The arrows indicate the start codon of coding regions for VP1 and VP2, respectively. The letters in bold represent the change of nucleotides for mammalian codon optimization. The numbers on the left indicate the positions of nucleotides in VP1 from the 5′ end to 3′ end.

FIGS. 6A-6E (GenBank: AY386330.1) show the sequence of the B19 virus isolate J35, complete genome (FIG. 6A). The p5 promoter is shown by underlining and the 3′ UTR is shown in bold. The amino acid sequences of NS1, 7.5 kDa protein, protein X, and 11 kDa protein are provided at FIGS. 6B-6E.

FIG. 7 shows the sequence of a mutant PLA2 sequence.

### DETAILED DESCRIPTION OF THE INVENTION

The invention features compositions and methods that are useful for producing a parvovirus immunogenic composition and methods of using such compositions for the treatment or prevention of parvovirus infection.

The invention is based, at least in part, on the discovery that codon usage is responsible for the cell type-specific expression of the viral capsid gene of human parvovirus B19 (B19V), which has an extreme tropism for human erythroid progenitors. This was surprising given that tissue specific expression is typically regulated by the promoter and/or the 3′ untranslated region. Based on this novel finding, the codon usage of B19V capsid genes was optimized for mammalian cell expression. Transfection with codon-optimized capsid genes into different mammalian cell lines, including 293T, Cos 7 and Hela, produced viral-like particles (VLPs). Accordingly, the invention provides codon-optimized capsid genes encoding VLPs, methods for producing such VLPs, cells and vector comprising the codon-optimized capsid genes, the use of such genes for the production of vaccines, and related methods for the prevention or treatment of parvovirus infections.

### Parvovirus Polynucleotides

In general, the invention includes any codon optimized nucleic acid molecule encoding a VLP comprising one or more parvovirus polypeptides or a fragments thereof, where the fragment induces an immune response and the codon optimized nucleic acid molecule is capable of expression in a nonpermissive cell type. Such codon optimized nucleic acid molecules need not be optimized in their entirety. For example, a codon optimized nucleic acid molecule may comprise at least about 50%-100% (e.g., 50%, 75%, 85%) optimized codons. Preferably, a nucleic acid molecule includes a sufficient number of optimized codons to permit expression of a parvovirus capsid or other structural protein in a nonpermissive cell type (e.g., a nonerythroid lineage cell). In particular embodiments, the codon optimized polynucleotide sequence has at least about 85%, 90%, 95% or more nucleic acid identity to the sequence shown at FIG. 5. In other embodiments, a polynucleotide of the invention comprises or consists essentially of the nucleic acid sequence shown at FIG. 5. Such polynucleotides are useful, for example, for the in vitro or in vivo expression of a VLP. Accordingly, the invention provides immunogenic compositions comprising such polynucleotides that are useful for subcutaneous vaccination (i.e., in the form of a DNA vaccine). In other embodiments, the invention provides immunogenic compositions comprising a VLP encoded by a polynucleotide of the invention. In still other embodiments, the invention provides immunogenic compositions comprising a combination of a polynucleotide of the invention and a VLP encoded by such polynucleotide. The polynucleotides of the invention can be administered concurrently with the VLP, or sequentially.

If desired, a polynucleotide of the invention is an isolated nucleic acid molecule. Such an isolated nucleic acid molecule can be manipulated by recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. In certain exemplary embodiments, the vector comprises codon optimized parvovirus nucleic acid segments, or fragments thereof (e.g., fragments of the sequence shown in FIG. 5). The vector may further comprise a CMV or B19 p6 promoter.

In addition, the nucleotides can be sequenced to ensure that the correct coding regions were cloned and do not contain any unwanted mutations. The nucleotides can be subcloned into an expression vector (e.g. pIRES) for expression in any cell.

An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, as the term is used herein, because it is readily manipulatable by standard techniques known to those of ordinary skill in the art.

### Parvovirus VLP Production

The invention also provides constructs comprising a codon optimized nucleic acid molecule and methods for producing a VLP comprising parvovirus polypeptides, or fragments thereof in a nonpermissive cell type, as well as compositions and methods that increase the efficiency of VLP production in such cells. In various embodiments, the codon optimized nucleic acid molecules are useful for in vitro or in vivo expression (i.e., expression in a human or canine subject having or at risk of developing a parvovirus infection). For example, the use of a p6 promoter or portions thereof in an expression vector comprising a codon optimized nucleic acid molecule of the invention, can improve the efficiency of parvovirus protein production in a cell. In another example, a 3′ UTR is included in the expression vector. A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

Constructs and/or vectors provided herein comprise codon optimized parvovirus polynucleotides that encode structural polypeptides, or portions thereof as described herein. The vector may be, for example, a phage, plasmid, viral, or retroviral vector. The constructs and/or vectors that comprise the nucleotides should be operatively linked to an appropriate promoter, such as the CMV promoter, phage lambda PL promoter, the E. coli lac, phoA and tac promoters, the SV40 early and late promoters, and promoters of retroviral LTRs are non-limiting examples. In one embodiment, the promoter is a parvovirus B19 p6 promoter. The constructs and/or vectors that comprise the nucleotides may also be operatively linked to an inducible promoter. The inducible promoter can be selected from any inducible promoter that is known in the art, including a tetracycline inducible promoter, e.g., T-REX™ (Invitrogen, Carlsbad, Calif.). Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated. If desired, the vector further comprises a 3′ UTR, such as a parvovirus B19 3′ UTR.

Expression vectors will typically include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. In particular embodiments, the vector is a bicistronic vector (e.g., pIRES). Other suitable vectors will be readily apparent to the skilled artisan.

Recombinant constructs can be prepared and used to transfect, infect, or transform and can express viral proteins, including those described herein, into eukaryotic cells and/or prokaryotic cells. Thus, the invention provides for host cells which comprise a vector (or vectors) that contain nucleic acids which code for parvovirus structural proteins in a host cell under conditions which allow the formation of VLPs.

The introduction of the recombinant constructs into the eukaryotic cells and/or prokaryotic cells can be a transient transfection, stable transfection, or can be a locus-specific insertion of the vector. Transient and stable transfection of the vectors into the host cell can be effected by any method known in the art, including, but not limited to, calcium phosphate transfection, DEAE-dextran mediated transfection, cationic lipid-mediated transfection, electroporation, transduction, and infection. Such methods are described in many standard laboratory manuals, such as Davis et al., Basic Methods in Molecular Biology (1986); Keown et al., 1990, Methods Enzymol. 185: 527-37; Sambrook et al., 2001, Molecular Cloning, A Laboratory Manual, Third Edition, Cold Spring Harbor Laboratory Press, N.Y., which are hereby incorporated by reference.

In another embodiment, the vector and/or host cell comprise nucleotides that encode parvovirus proteins, or portions thereof as described herein. In another embodiment, the vector encodes a protein that consists essentially of parvovirus or Parvovirus B19 (B19V) structural proteins VP1 or VP2, or portions thereof as described herein. In a related embodiment, the vector encodes a VP1 protein containing an altered PLA2 motif that lacks or has reduced inflammatory properties when injected into a subject relative to a wild-type PLA2 motif. Examples of such PLA2 motif mutations include, but are not limited to, a PLA2 deletion, a P133R mutation, an H153A mutation, and a D175A mutation. PLA2 mutations, as well as methods for making and using VP1 proteins having an altered PLA2 motif, are described in Lu et al., J. Infect. Dis. 193:582-590 (2006) and Filippone et al., Virology 374:444-452 (2008), which are hereby incorporated by reference.

Once a recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

Methods to grow cells that produce VLPs of the invention include, but are not limited to, batch, batch-fed, continuous and perfusion cell culture techniques. In one embodiment, a cell comprising a codon optimized parvovirus nucleic acid molecule is grown in a bioreactor or fermentation chamber where cells propagate and express protein (e.g. recombinant proteins) for purification and isolation. Typically, cell culture is performed under sterile, controlled temperature and atmospheric conditions. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 1000 L bags.

The VLPs are isolated using methods that preserve the integrity thereof, such as by gradient centrifugation, e.g., cesium chloride, sucrose and iodixanol, as well as standard purification techniques including, e.g., ion exchange and gel filtration chromatography. The following is an example of how VLPs of the invention can be made, isolated and purified. A person of skill in the art appreciates that there are additional methods that can be used to make and purify VLPs. Accordingly, the invention is not limited to the methods described herein.

### Parvovirus Polypeptides and Analogs

The invention provides VLPs comprising one or more parvovirus polypeptides. Also included in the invention are VLPs comprising one or more parvovirus polypeptides or fragments thereof that are modified in ways that enhance or do not inhibit their ability to modulate an immune response or that enhance or do not inhibit their expression in a nonpermissive cell type. In one embodiment, the invention provides methods for optimizing a parvovirus amino acid sequence or nucleic acid sequence by producing an alteration. In particular, the invention provides an optimized nucleic acid molecule shown at FIG. 5. If desired, that optimized nucleic acid molecule includes one or more additional alterations. Such alterations may include certain mutations, deletions, insertions, or post-translational modifications. The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 10, 13, 15 amino acid residues, preferably at least 25 amino acid residues, and more preferably more than 35 amino acid residues.

Alterations of a parvovirus polypeptide or polynucleotide include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis is also included in the present invention. In one embodiment, mutagenesis can be guided by known information of the naturally occurring molecule or altered or mutated naturally occurring molecule, e.g., sequence, sequence comparisons, physical properties, crystal structure or the like.

In one embodiment, the invention provides polypeptide variants that differ from a reference polypeptide. The term “variant” refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. The polynucleotides encoding such variants comprises a codon optimized sequence. Preferably, a parvovirus nucleic acid molecule of the invention includes at least about 50%, 60%, 75%, 80%, 90%, 95% or even 100% optimized codons. Guidance in determining which amino acid residues can be substituted, inserted, or deleted without eliminating biological or immunological activity can be found using computer programs well known in the art, for example, DNASTAR software. Desirably, variants show substantial biological activity. In one embodiment, a protein variant forms a VLP and elicits an antibody response when administered to a subject.

Natural variants can occur due to mutations in the proteins. These mutations may lead to antigenic variability within individual groups of infectious agents, for example parvovirus. Thus, a person infected with a particular strain develops antibody against that virus, as newer virus strains appear, the antibodies against the older strains no longer recognize the newer virus and reinfection can occur. The invention encompasses all antigenic and genetic variability of proteins from infectious agents for making VLPs.

Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or gamma amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs having a chemical structure designed to mimic parvovirus VLPs or one or more parvovirus polypeptides functional activity can be administered according to methods of the invention. Parvovirus polypeptide analogs may exceed the physiological activity (e.g., immunogenicity) of native parvovirus. Methods of analog design are well known in the art, and synthesis of analogs can be carried out according to such methods by modifying the chemical structures such that the resultant analogs exhibit the immunomodulatory activity of a native parvovirus polypeptide. These chemical modifications include, but are not limited to, substituting alternative R groups and varying the degree of saturation at specific carbon atoms of the native parvovirus molecule. Preferably, the analogs are relatively resistant to in vivo degradation, resulting in a more prolonged therapeutic effect upon administration. Assays for measuring functional activity include, but are not limited to, those described in the Examples below.

### Immunogenic Compositions

The invention provides compositions and methods for inducing an immunological response in a subject, particularly a human, which involves inoculating the subject with a codon optimized nucleic acid molecule encoding a VLP, a VLP comprising one or more parvovirus polypeptides, or fragments thereof, or a combination thereof, in a suitable carrier for the purpose of inducing or enhancing an immune response. In one embodiment, an immune response protects the subject from a parvovirus infection. The administration of this immunological composition may be used either therapeutically in subjects already experiencing a parvovirus infection, or may be used prophylactically to prevent a parvovirus infection.

The preparation of immunogenic compositions and vaccines is known to one skilled in the art. In one embodiment, the vaccine includes a VLP comprising one or more parvovirus polypeptides, or fragments thereof. In another embodiment, the invention provides an expression vector encoding one or more parvovirus polypeptides or fragments thereof or variants thereof. Such an immunogenic composition is delivered in vivo in order to induce or enhance an immunological response in a subject, such as a humoral response.

For example, a VLP comprising one or more parvovirus polypeptides, or fragments or variants thereof are delivered in vivo in order to induce an immune response.

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the subject receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

The VLP comprising one or more parvovirus, or fragments or variants thereof may be administered in combination with an adjuvant. Adjuvants are immunostimulating agents that enhance vaccine effectiveness. If desired, the VLP comprising one or more parvovirus polypeptides or fragments or variants thereof are administered in combination with an adjuvant that enhances the effectiveness of the immune response generated against the antigen of interest. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the VLP comprising one or more parvovirus polypeptides, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The immunogenic compositions are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

Immunogenic compositions are administered in a manner compatible with the dose formulation. The immunogenic composition comprises an immunologically effective amount of the VLP and other previously mentioned components. By an immunologically effective amount is meant a single dose, or a composition administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgment of the practitioner, but typically range between 5 μg to 250 μg of antigen per dose.

The invention provides a VLP for use in treating or preventing a parvovirus infection (e.g., Parvovirus B19 (B19V)). In particular, the present invention provides methods of treating viral diseases and/or disorders or symptoms thereof which comprise administering a therapeutically effective amount of a pharmaceutical composition comprising a codon optimized nucleic acid molecule encoding a VLP or a VLP produced in a nonpermissive cell type using a codon optimized parvovirus nucleic acid molecule herein to a subject (e.g., a mammal such as a human). Thus, one embodiment is a method of treating a subject suffering from or susceptible to a viral infection, viral disease or disorder or symptom thereof. The method includes the step of administering to the mammal a therapeutic or prophylactic amount of an amount of a compound herein sufficient to treat the disease or disorder or symptom thereof, under conditions such that the disease or disorder is prevented or treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the agents herein, such as a VLP of a formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which a parvovirus may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with parvovirus, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pharmaceutical Compositions and Administration

The invention features pharmaceutical compositions that comprise codon optimized nucleic acid molecules encoding a VLP and/or VLPs produced using the optimized nucleic acid molecules described herein. The pharmaceutical compositions useful herein contain a pharmaceutically acceptable carrier, including any suitable diluent or excipient, which includes any pharmaceutical agent that does not itself induce the production of an immune response harmful to the vertebrate receiving the composition, and which may be administered without undue toxicity and a VLP of the invention. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate.

In particular embodiments, the invention encompasses an antigenic formulation comprising a codon optimized nucleic acid molecule of the invention and/or VLPs which comprises at least one viral protein, for example one parvovirus protein produced by expressing a codon optimized nucleic acid molecule. In certain preferred embodiments, the pharmaceutical composition comprises VLPs of parvovirus, and a pharmaceutically acceptable carrier. In other certain preferred embodiments, the pharmaceutical composition comprises VLPs of parvovirus, an adjuvant, and a pharmaceutically acceptable carrier.

In one embodiment, the VLPs are comprised of parvovirus structural proteins VP2 and VP1. Preferably, the VLP comprises VP2 and VP1 in a ration of about 75:25, 80:20, or 90:10, 95:5. Preferably the VP2:VP1 ratio is 95:5. In another embodiment, the pharmaceutical composition further comprises a parvovirus protein. The parvovirus protein is, in certain examples, a structural protein. The invention also encompasses a vaccine formulation comprising VLPs that comprise at least one viral protein, for example a VP1 or VP2 protein. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

In certain embodiments, the VLP or polynucleotide composition is supplied in liquid form, for example in a sealed container indicating the quantity and concentration of the VLP composition. Preferably, the liquid form of the VLP composition is supplied in a hermetically sealed container at least about 50 μg/ml, more preferably at least about 100 μg/ml, at least about 200 μg/ml, at least 500 μg/ml, or at least 1 mg/ml.

Generally, VLP and/or polynucleotide vaccines of the invention are administered in an effective amount or quantity (as described herein) sufficient to stimulate an immune response against one or more strains of a virus a described here, for example, Parvovirus B19 (B19V). Preferably, administration of the VLP and/or polynucleotide of the invention elicits immunity against a parvovirus. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract or small particle aerosol (less than 10 microns) or spray into the lower respiratory tract.

Thus, the invention also comprises a method of formulating a vaccine or antigenic composition that induces immunity to an infection or at least one symptom thereof to a mammal, comprising adding to the formulation an effective dose of VLPs, e.g. parvovirus VLP and/or polynucleotides encoding such VLPs. In one embodiment, the infection is a Parvovirus B19 (B19V) infection.

In certain cases, stimulation of immunity with a single dose is preferred, however additional dosages can be also be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections. Similarly, adults who are particularly susceptible to repeated or serious infections, such as, for example, health care workers, day care workers, family members of young children, the elderly, and individuals with compromised cardiopulmonary function or immune systems may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Prime Boost

The present methods also include a variety of prime-boost regimens. In these methods, one or more priming immunizations is followed by one or more boosting immunizations. The actual immunogenic composition can be the same or different for each immunization and the route, and formulation of the immunogens can also be varied. For example, the prime-boost regimen can include administration of an immunogenic composition comprising a VLP encoded by a polynucleotide of the invention alone or in combination with a codon optimized nucleic acid molecule of the invention. Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. The dosages can be determined from animal studies. A non-limiting list of animals used to study the efficacy of vaccines include the guinea pig, hamster, ferrets, chinchilla, mouse and cotton rat, and non-human primates. Most animals are not natural hosts to infectious agents but can still serve in studies of various aspects of the disease. For example, any of the above animals can be dosed with a vaccine candidate, e.g. VLPs of the invention, to partially characterize the immune response induced, and/or to determine if any neutralizing antibodies have been produced. In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

The VLPs of the invention can also be formulated with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the VLPs, or can be administered separately. Either the protein or an expression vector encoding the protein can be administered to produce an immunostimulatory effect. Thus in one embodiment, the invention comprises antigenic and vaccine formulations comprising an adjuvant and/or an immune stimulator.

### Methods of Delivery

The codon optimized nucleic acid molecules and VLPs of the invention are useful for preparing compositions that stimulate an immune response. Such compositions are useful for the treatment or prevention or a viral infection (e.g., a parvovirus infection). Both mucosal and cellular immunity may contribute to immunity to infectious agents and disease. In one embodiment, the invention encompasses a method of inducing immunity to a viral infection, for example parvovirus infection in a subject, by administering to the subject a parvovirus virus VLP.

The invention also provides a method to induce immunity to viral infection or at least one symptom thereof in a subject, comprising administering at least one effective dose of a codon optimized nucleic acid molecule and/or a VLP as described herein, for example a VLP comprising one or more viral proteins, for example one or more parvovirus proteins. In certain cases, the VLP further comprises VP1 and/or VP2. In another embodiment, the method comprises inducing immunity to a viral infection, e.g. parvovirus infection or at least one symptom thereof by administering the formulation in multiple doses.

Codon optimized nucleic acid molecules and/or VLPs of the invention can induce substantial immunity in a vertebrate (e.g. a human) when administered to the vertebrate. The substantial immunity results from an immune response against VLPs of the invention that protects or ameliorates infection or at least reduces a symptom of infection in the vertebrate. In some instances, if the vertebrate is infected, the infection will be asymptomatic. The response may be not a fully protective response. In this case, if the vertebrate is infected with an infectious agent, the vertebrate will experience reduced symptoms or a shorter duration of symptoms compared to a non-immunized vertebrate.

In one embodiment, the invention comprises a method of inducing substantial immunity to parvovirus infection or at least one symptom thereof in a subject, comprising administering at least one effective dose of a codon optimized nucleic acid molecule of the invention and/or a VLP. In particular embodiments, the infection is parvovirus and the codon optimized nucleic acid molecule encodes a VLP that comprises one or more parvovirus envelope protein as described herein. In another embodiment, the invention comprises a method of vaccinating a mammal against a parvovirus comprising administering to the mammal a protection-inducing amount of a codon optimized nucleic acid molecule of the invention alone or in combination with a VLP comprising at least one parvovirus protein.

As mentioned above, the VLPs of the invention prevent or reduce at least one symptom of an infection in a subject. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of viral infection or additional symptoms, a reduced severity of viral symptoms or a suitable assays (e.g. antibody titer and/or T-cell activation assay). The objective assessment comprises both animal and human assessments.

### Kits

The invention also provides for a pharmaceutical pack or kit comprising one or more containers filled with one or more of the ingredients of the polynucleotide and or VLP vaccine formulations of the invention. In a preferred embodiment, the kit comprises two or more containers, one containing VLPs, another containing a codon optimized nucleic acid molecule and, optionally, another containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the codon optimized nucleic acid molecules and/or VLP formulations be packaged in a hermetically sealed container such as an ampoule or sachette indicating the quantity of composition. In one embodiment, the codon optimized nucleic acid molecule and/or VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

The invention also features a kit comprising a codon optimized nucleic acid molecule and/or VLP as described herein and instructions for use in an immunization method delineated herein.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### The B19V Capsid Gene is Expressed in a Cell-Type Specific Manner

Parvovirus B19 (B19V) has a small (22 nm), nonenveloped, icosahedral capsid encapsidating a single-stranded DNA genome of 5,596 nucleotides. Transcription of the B19V genome is controlled by a single promoter (p6), which is located at map unit 6 and regulates the synthesis of all nine viral transcripts. There is a single non-spliced transcript for the production of the nonstructural protein (NS1), and eight transcripts generated by a combination of different splicing events, encoding two capsid proteins (VP1 and VP2), and two smaller proteins (7.5 kDa and 11 kDa) of unknown function. In addition, a short open reading frame (ORF) (encoding a putative “X protein”) was observed in the VP1 coding region of B19V. Although there is no evidence showing that this small ORF is expressed in B19V, it is structurally similar to the SAT protein that was characterized in porcine parvovirus.

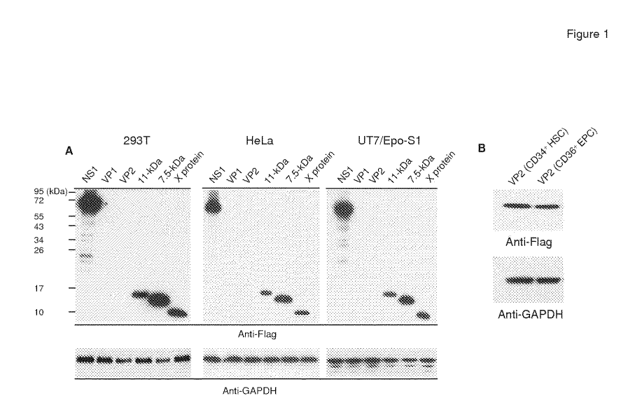

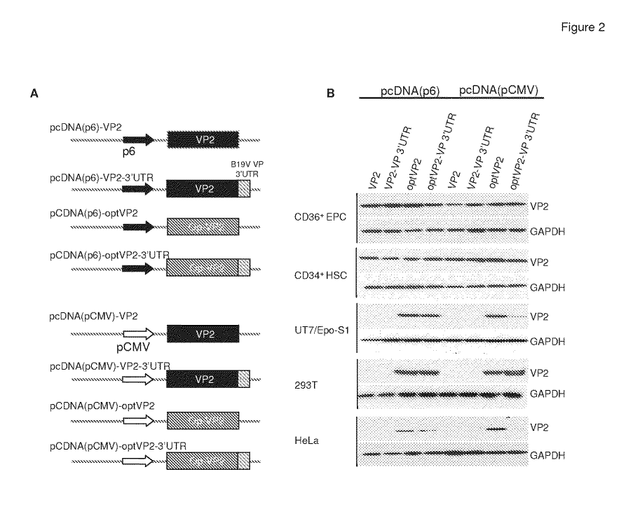

In order to evaluate the expression of B19V genes in different cell lines, as well as primary CD34⁺ hematopoietic stem cells (HSCs) and CD36⁺ erythroid progenitor cells (EPCs), the genes encoding NS1, VP1, VP2, 11-kDa, 7.5 kDa, and protein X, were cloned into pCMV-3Tag-6, in which the expression of these viral genes is controlled by a CMV early promoter. Several non-permissive or semi-permissive cell lines, including 293T, HeLa and UT7/Epo-S1 cells, were transfected with recombinant plasmids composed of NS1, VP1, VP2, 11-kDa, 7.5 kDa, and protein X, and the expression of these viral genes was examined by immunoblot analysis. At 48 hours post-transfection (hpt), the proteins of NS1, 11-kDa, 7.5-kDa, and protein X were detected in the three different cell lines tested, but VP1 and VP2 proteins were undetectable under identical conditions (FIG. 1A). To determine if B19V capsid gene could be expressed in CD34⁺HSCs or CD36⁺EPCs, the cells were transfected with the recombinant plasmid composed of the VP2 gene. At 48 hpt, VP2 protein was detected in both CD34⁺ HSCs and CD36⁺ EPC (FIG. 1B). Taken together, these results indicated a cell type-specific expression of B19V capsid gene.

### Example 2

### Codon-Optimized VP2 was Expressed in Non-Permissive Cell Lines

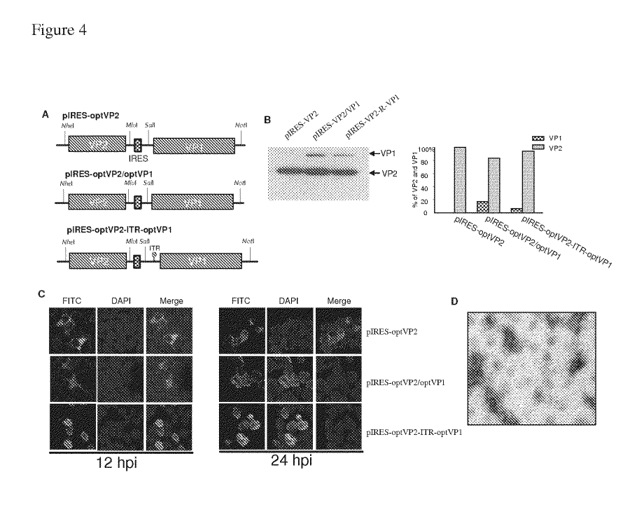

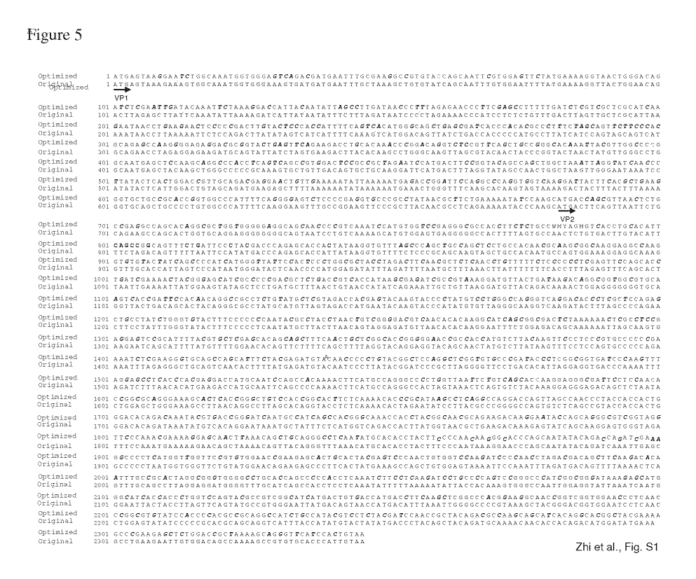

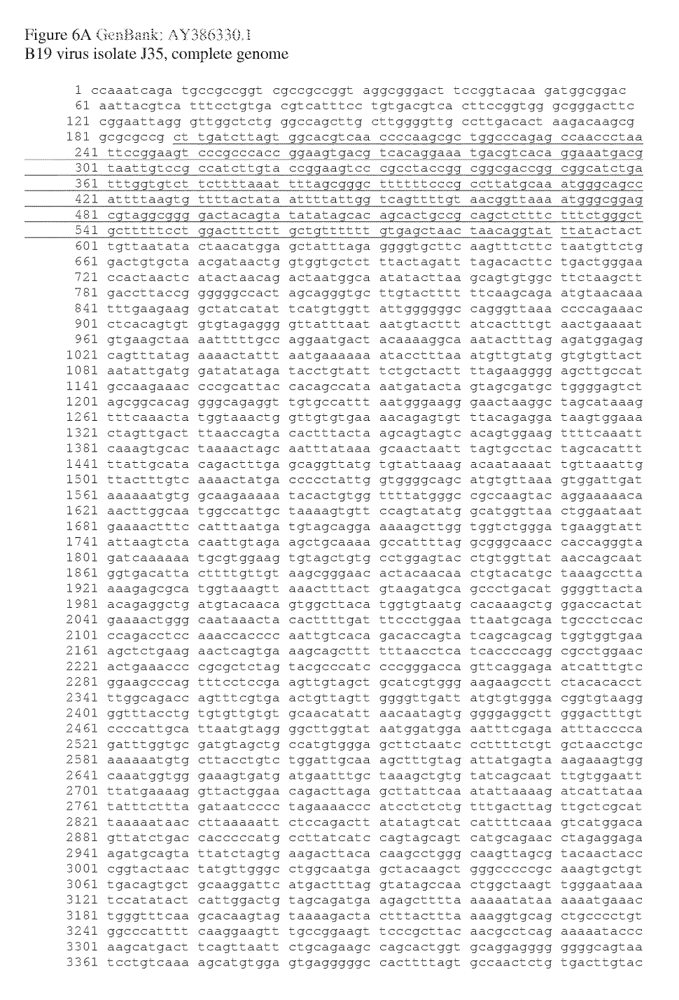

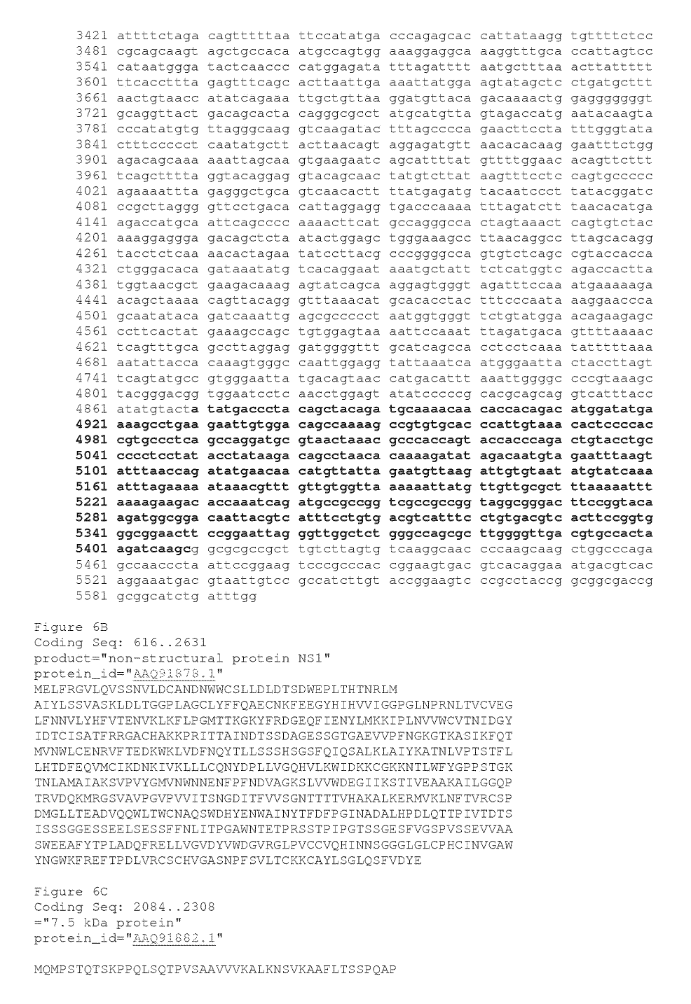

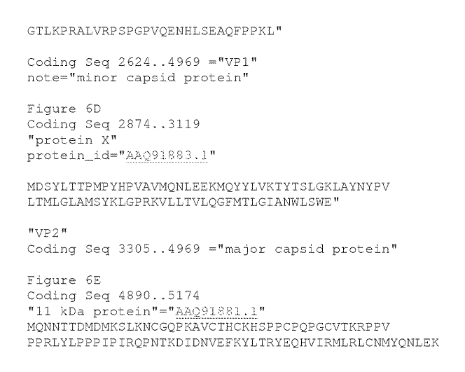

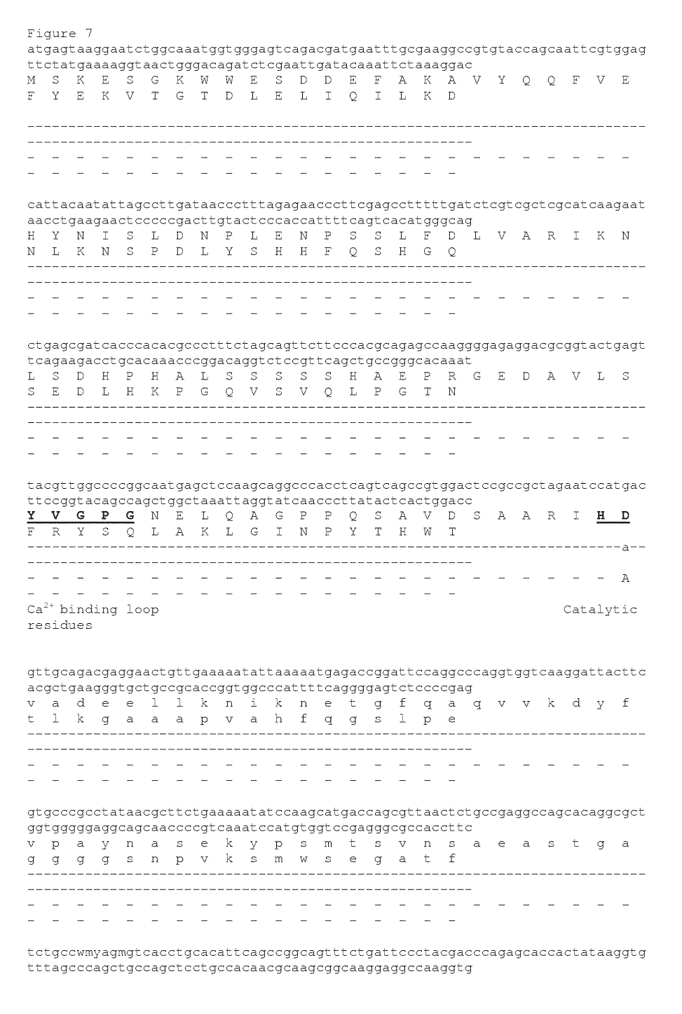

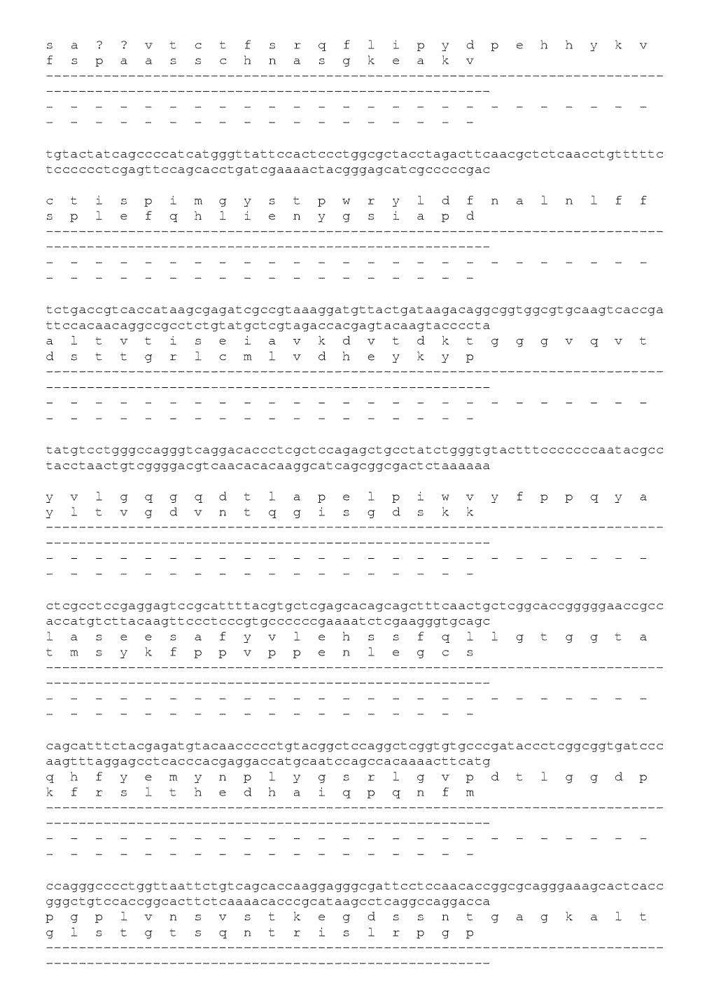

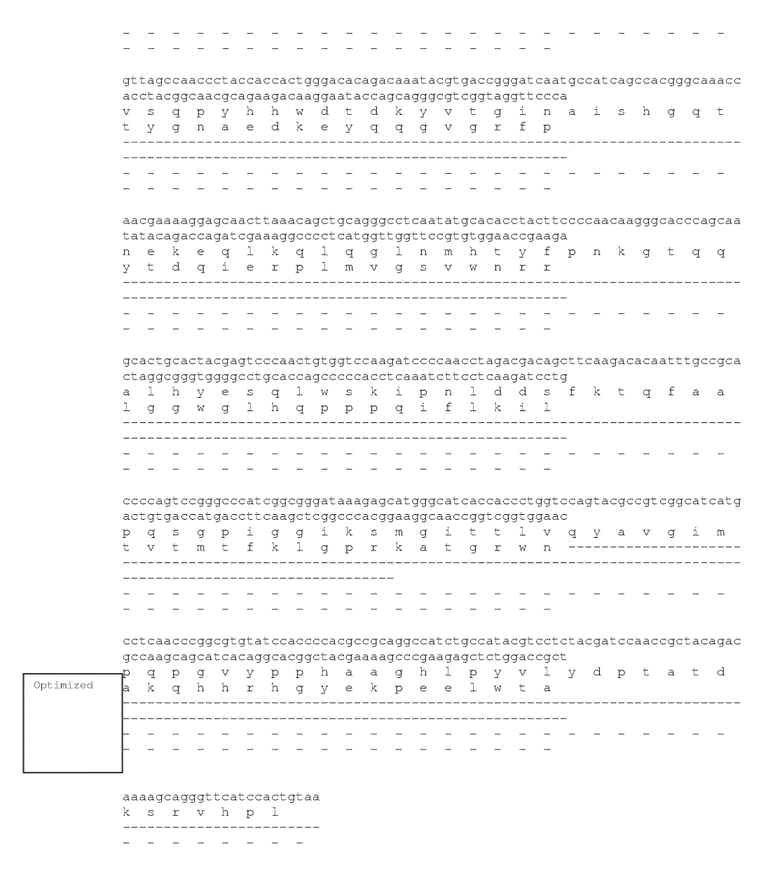

In order to confirm the observation that the expression of viral capsid is cell type-specific, as well as to improve the expression of viral capsid genes in cell lines that were regularly used in the laboratory, the entire open reading frame of the VP2 capsid gene and VP1 unique region (VP1u) (Genbank AY386330) were synthesized and codon-optimized for mammalian codon usage by the Celtek Bioscience, LLC. (Nashville, Tenn.) (FIG. 5). Synthesized fragments encompassing the full-length VP2 gene or VP1u were cloned into pcDNA3.1. The full-length VP1 gene was obtained by overlapping PCR using the synthesized fragments of VP2 and VP1u as templates. In addition, to determine the possible contribution of the promoter and the 3′ untranslated region (3′UTR) to the cell type-specific expression of B19V capsid genes, a series of recombinant plasmids was constructed (FIG. 4A): i) expression of wild-type or codon-optimized VP2 gene was controlled by either B19V p6 promoter or pCMV early promoter; ii) the open reading frame of wild-type or codon-optimized VP2 was linked to B19V VP 3′UTR or directly to a SV40 early polyadenylation (poly(A)). HeLa, 293T, UT7/Epo-S1, CD34⁺ HSCs, and CD36⁺ EPC were transfected with the respective plasmids and the capsid protein synthesis was examined by immunoblotting with antibody specific for B19V VP2. As shown in FIG. 2, in HeLa, 293T, and UT7/Epo-51 cells, production of VP2 protein was only detected in cells transfected with plasmids carrying codon-optimized VP2 genes, including pcDNA(p6)-OptVP2, pcDNA(p6)-OptVP2-3′UTR, pcDNA(pCMV)-OptVP2, and pcDNA(pCMV)-OptVP2-3′UTR. Production of VP2 protein was not detected in those cells transfected with wild-type VP2 genes, including pcDNA(p6)-VP2, pcDNA(p6)-VP2-3′UTR, pcDNA(pCMV)-VP2, and pcDNA(pCMV)-VP2-3′UTR. However, in the case of erythroid lineage cells, CD34⁺ HSCs and CD36⁺ EPCs, production of VP2 was detected in all samples tested regardless of codon usage. These results indicate that the codon-optimized VP2 gene was able to be expressed in non-permissive or semi-permissive cell lines. Interestingly, in HeLa and UT7/Epo-S1 cells, VP2 production in the cells transfected with transfected pcDNA(pCMV)-OptVP2-3′UTR was significantly less than those with pcDNA(pCMV)-OptVP2, but there was no significant difference between pcDNA(p6)-OptVP2 and pcDNA(p6)-OptVP2-3′UTR-transfected cells. Furthermore, the VP2 gene was highly expressed in the 293T cells transfected with either pcDNA(pCMV)-VP2 or pcDNA(pCMV)-VP2-3′UTR. These results indicate that the 3′UTR of B19V VP mRNA inhibited the activity of CMV early promoter, but had less effect on its own p6 promoter. This negative impact was not apparent in 293T cells.

### Example 3

### Codon-Optimization Likely Improved VP2 Protein Translation

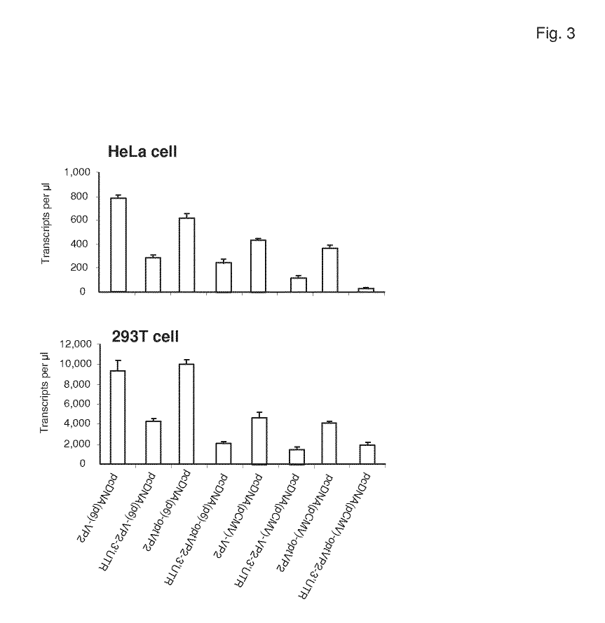

In order to quantitatively assess the effect of codon optimization on the transcription of the VP2 gene, as well as the effect of other factors, such as the promoter and 3′UTR, real-time RT-PCR was performed to compare the level of RNA transcripts of wild-type and codon-optimized VP2 in HeLa and 293T cells. As shown in FIG. 3, transfection of wild-type or codon-optimized VP2 into HeLa and 293T cells yielded measurable amounts of VP2 mRNA. Therefore, at the transcriptional level, there was no significant difference between wild-type and codon-optimized VP2 in HeLa and 293T. Without wishing to be bound by theory, these results likely indicate that the enhanced production of VP2 detected by immunoblotting was probably due to an improvement of translation by codon optimization. In comparison with pCMV promoter, under the same conditions, the mRNA level of VP2 was significantly higher when the B19 p6 promoter was used (p<0.001). A previous study had shown that the 3′UTR of B19 capsid mRNA inhibits its own mRNA translation in nonpermissive cells. Real-time RT-PCR showed that the level of VP2 mRNA was always lower in cells transfected with the plasmids containing B19V VP 3′UTR (pcDNA(p6)-VP2-3′UTR, pcDNA(p6)-OptVP2-3′UTR, pcDNA(pCMV)-VP2-3′UTR and pcDNA(pCMV)-Opt), regardless of whether the p6 or the pCVM promoter was used. Consistent with the immunoblot results, these results indicated that B19V VP 3′UTR has a negative impact on both the p6 and the pCMV. Moreover, overall transcription levels of B19V capsid gene were at least 10 fold higher in 293T cells than in HeLa cells.

### Example 4

### Non-Erythroid Progenitor Cells Transfected with pIRES-Op-VP2-ITR-VP1 Produced Typical Parvovirus-Like Particles

In an attempt to optimize the experimental conditions for production of B19V VLP in mammalian cell line that was commonly used in the laboratory, codon-optimized VP1 and VP2 genes were subcloned into a bicistronic expression vector pIRES (Supplementary Materials and Methods). In addition, the pIRES vector was modified by inserting an inverted-repeat (ITR) sequence immediately upstream of the VP1 gene to further adjust the ratio of VP1 versus VP2 (FIG. 4A). 93T cells were transfected with pIRES-Opt-VP2, pIRES-Opt-VP2/VP1, and pIRES-Op-VP2-ITR-VP1 and the expression of capsid genes was examined by immunoblot analysis. As shown in FIG. 4B, bands with the appropriate molecular mass of VP1 or VP2 were detected in the transfected 293T cells. The expression levels of the VP2 gene were similar among the three transfected samples, whereas production of VP1 was not detected in the cells with pIRES-Opt-VP2, and different between pIRES-Opt-VP2/VP1 and pIRES-Opt-VP2-ITR-VP1. The VP1: VP2 ratios in the cells transfected with pIRES-Opt-VP2/VP1 or pIRES-Opt-VP2-ITR-VP1 were 1:5 and 1:20, respectively. The formation of viral capsid in the cells transfected with different plasmids was examined by immunofluorescent staining with MAb 521-5D, which recognizes a conformational epitope in the VP2 region (FIG. 4C). At 12 hpt, capsid proteins were detected mainly in the cytoplasm of cells transfected with the pIRES-Opt-VP2. In contrast, capsid proteins were predominantly detected in the nucleus of the cells transfected with pIRES-Opt-VP2/VP1 and pIRES-Opt-VP2-ITR-VP1.

In contrast to pIRES-Opt-VP2/VP1-transfected cells, the appearance of viral capsids in the pIRES-Opt-VP2-ITR-VP1-transfected cells more closely resembled a natural B19V infection. Formation of small clusters that were evenly distributed in the nucleus was observed. At 24 hpt viral capsid proteins were detected in both the cytoplasm and the nucleus of the cells transfected with all three different plasmids. Since the ratio of VP1:VP2 and conformation of viral capsid in the pIRES-Opt-VP2-ITR-VP1-transfected cells was more similar to those of natural B19V infection, pIRES-Opt-VP2-ITR-VP1 was employed for production of VLP in 293T cells. When cell lysates were subjected to sequential sedimentation in sucrose and CsCl, banding of parvovirus proteins (determined by immunoblot) was detected at 1.31 g/ml, the density of empty capsids. Direct electron microscopy of harvests from cultures transfected with pIRES-Op-VP2-ITR-VP1 revealed typical parvovirus-like particles (FIG. 4D).

In summary, cell type-specific expression of wild-type viral capsid genes is reported herein. The cell-type specific expression was conferred by codon usage, rather than by the promoter and 3′UTR. Thus, codon usage is the key factor regulating the preferential expression of B19V capsid genes in erythroid progenitors. Based on this novel finding, the codon usage of B19V capsid genes was optimized for mammalian cell expression. Transfection of codon-optimized capsid genes into mammalian cell lines produced viral-like particles. These results provide for the straightforward production of B19V VLP in a variety of non-erythroid mammalian cells. Accordingly, the invention features codon-optimized parvovirus polynucleotides, mammalian expression vectors comprising such polynucleotides, and cells expressing such vectors, and methods of using these compositions for the production of a B19V vaccine or other immunogenic composition.

The results reported herein were carried out using the following methods and materials.

### Plasmid Construction

To construct recombinant plasmids vectors that comprised of B19V NS1, VP1, VP2, 11-kDa, 7.5 kDa, protein X, the open reading frames of respective genes were amplified by PCR from the B19V infectious clone, pB19-M20, constructed in Zhi et al., (2004. Virology 318:142-152) and then cloned into pCMV-3Tag-6 (Stratagene, La Jolla, Calif.) with 3× Flag epitopes at the NH₂ terminus, generating pCMV-FlagNS, pCMV-FlagVP1, pCMV-FlagVP2, pCMV-Flag11 kDa, pCMV-Flag7.5 kDa pCMV-FlagX.

A pcDNA3.1 vector with a human cytomegalovirus immediate-early promoter (pCMV) and a SV40 early polyadenylation signal was obtained from Invitrogen (Invitrogen, Carlsbad, Calif.). To construct a pcDNA(p6) plasmid carrying a B19V p6 promoter, the p6 promoter region (nt 188-584) of the B19V J35 strain (accession no: AY386330) was amplified by PCR using adequate forward and reverse primers carrying NruI and HindIII restriction sites at their 5′ ends, respectively, and inserted between NruI and HindIII sites of a pcDNA3.1 vector (Invitrogen), resulting in replacement of a human cytomegalovirus promoter (pCMV) with the p6 promoter. A VP2-coding sequence (plus a stop codon; nt 3305-4969) or a sequence spanning VP2-coding and its 3′ UTR (nt 3305-nt 5409) regions was amplified by PCR using a B19V J35 strain as a template with proper forward and reverse primers hanging HindIII (plus a Kozak sequence) and XhoI sites at their 5′ ends, respectively. VP2 or VP2 plus 3′UTR DNA fragments were inserted into respective sites of pcDNA(p6) and pcDNA3.1, generating plasmids termed pcDNA(p6)-VP2, pcDNA(p6)-VP2-3′UTR, pcDNA(pCMV)-VP2, and pcDNA(pCMV)-VP2-3′UTR.

A codon-optimized VP2 (optVP2)-coding (plus a stop codon) sequence hanging HindIII (+ a Kozak sequence) and XbaI sites at their 5′ ends, respectively, was inserted into respective sites of pcDNA3.1-p6 and pcDNA3.1, resulting in pcDNA(p6)-optVP2 and pcDNA(pCMV)-optVP2. To create pcDNA(p6)-optVP2-3′UTR and pcDNA(pCMV)-optVP2-3′UTR, the 3′UTR (nt 4970-nt 5409) of B19V J35 strain was amplified using adequate forward and reverse primers with XbaI sites at their 5′ ends, followed by insertion into XbaI sites of pcDNA(p6)-optVP2 and pcDNA(pCMV)-optVP2 with the correct orientation.

In an attempt to bicistronically express B19V capsid genes, VP1 and VP2 genes were amplified by PCR and cloned into pIRES vector (Clonetech, Mountain View, Calif.) at the multiple cloning sites B and A, respectively. To control the ratio of VP1 versus VP2 in the VLP, an inverted repeat sequence (5′-GGATCCCGACGATCC-3′) was inserted in the 5′ untranslated region of the VP1 gene.

### Quantitative RT-PCR

RNA transcripts were quantitated by real-time RT-PCR as described in a previous study ¹⁹. Briefly, cells were harvested and total RNA extracted using Trizol (Invitrogen). RNA was reverse transcribed by initially incubating RNA with random primers and reverse transcriptase, Superscript II (Invitrogen). Quantitative PCR was performed with using the PerfeCta™ Multiplex qPCR SuperMix (Quant Biosciences) with primers and probes targeting the transcripts of wild-type VP2 (primers: 5′-CCTGGGCAAGTTAGCGTAC-3′ and 5′ATGAATCCTTGCAGCACTGTCA-3′; probe: 5′FAMTA-TGTTGGGCCTGGCAA-BHQ13′) or codon-optimized VP2 (primers: 5′CCCGTCAAATCCATGTGGTC3′ and 5′-AGTGGTGCTCTGGGTCGTAG-3′; probe: 5′FAM-CTTCTCTGCCAACAGCGTCA IABKFQ3′). After an initial activation step of 15 min at 95 C, 45 cycles of 15 s at 94° C. and 60 s at 60° C. were performed. The number of transcripts was quantified by estimating the cDNA copy number in compared to a standard curve of serial dilutions of pYT103 or codon-optimized VP2 plasmid. To confirm the extraction of RNA, and to normalize the numbers of transcripts per cell, quantitative RT-PCR was performed using the same amplification conditions, but with primers β-actin F (5′-GGCACCCAGCACAATGAAG-3′), 13 actin R (5′-GCCGATCCACACGGAGTACT-3′) and actin probe (5′ MAX550-TCAAGATCATTGCTCCTCCTGAGCGC-3′ IABLK). An actin standard curve was obtained from serial dilutions of a plasmid containing an extended region of the actin coding.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A nucleic acid molecule encoding a parvovirus structural protein or fragment thereof, wherein at least about 50-100% of the nucleic acid molecule's codons are optimized for expression in a nonpermissive mammalian cell.

2-3. (canceled)

4. The nucleic acid molecule of claim 1, wherein the parvovirus structural protein or fragment thereof is a parvovirus B19 (B19V) structural protein or fragment thereof, and wherein the nonpermissive mammalian cell is a mammalian non-erythroid lineage cell.

5. The nucleic acid molecule of claim 4, wherein the non-erythroid lineage cell is selected from the group consisting of 293T cells, COS cells, HeLa cells and UT7/Epo-S1 cells.

6. The nucleic acid molecule of claim 4, wherein the structural protein is:
i) a capsid protein;
ii) a VP1 or VP2;
iii) a VP1 or VP2 having at least about 85% amino acid identity to the sequence provided at NCBI Accession No. AAQ91879.1 (SEQ ID NO:1) and AAQ91880.1 (SEQ ID NO:2), respectively;
iv) a VP1 protein comprising an altered PLA2 motif that lacks or has reduced inflammatory properties when injected into a subject relative to a wild-type PLA2 motif;
v) a VP1 protein comprising an altered PLA2 motif selected from the group consisting of: a PLA2 deletion, a H153A mutation, a D175A mutation, and a P133R mutation; or
vi) a human B19V VP1 or VP2.

7-10. (canceled)

11. An expression vector comprising the nucleic acid molecule of claim 1.

12-15. (canceled)

16. The expression vector of claim 11, wherein the vector is selected from the group consisting of pcDNA(p6)-OptVP2, pcDNA(p6)-OptVP2-3′UTR, pcDNA(pCMV)-OptVP2, and pcDNA(pCMV)-OptVP2-3′UTR.

17. A mammalian expression vector comprising a CMV promoter positioned to control the expression of a first nucleic acid molecule encoding a parvo VP2 polypeptide and a second nucleic acid molecule encoding a parvo VP1 polypeptide, wherein the second nucleic acid molecule is separated from the first nucleic acid molecule by one or more inverted repeats.

18-24. (canceled)

25. The mammalian expression vector of claim 17, wherein the vector is selected from the group consisting of pIRES-Opt-VP2, pIRES-Opt-VP2/VP1, and pIRES-Op-VP2-ITR-VP1.

26-27. (canceled)

28. A cell comprising the expression vector of claim 11.

29-30. (canceled)

31. A method of producing a virus like particle comprising introducing into a nonpermissive or non-erythroid mammalian cell the expression vector of claim 11; culturing the cell under conditions to produce the structural proteins and form the VLP; and isolating the VLP.

32-34. (canceled)

35. An immunogenic composition comprising the nucleic acid molecule of claim 1.

36-40. (canceled)

41. The immunogenic composition of claim 35, further comprising an effective amount of a VLP comprising human B19V VP1 and VP2.

42. An immunogenic composition comprising an effective amount of the VLP of claim 31.

43. The immunogenic composition of claim 42, further comprising a nucleic acid molecule encoding a parvovirus B19 (B19V) structural protein or fragment thereof, wherein the codons of the nucleic acid molecule are optimized for expression in a mammalian non-erythroid lineage cell.

44-48. (canceled)

49. A kit comprising an effective amount of the nucleic acid molecule of claim 1 and instructions for the use of said kit in treating or preventing parvovirus infection.

50-53. (canceled)

54. The expression vector of claim 11, wherein the expression vector further comprises a tetracycline inducible promoter.

55. (canceled)

56. A method for producing an immune response in a subject, the method comprising administering to the subject an effective amount of the immunogenic composition of claim 35, thereby generating an immune response in said subject.

57. A method for producing an immune response in a subject, the method comprising administering to the subject an effective amount of the immunogenic composition of claim 42, thereby generating an immune response in said subject.

58. A method for treating or preventing a parvovirus infection in a subject, the method comprising administering to the subject an effective amount of the immunogenic composition of claim 35, and generating an immune response in said subject, wherein the immune response prevents or treats a parvovirus infection.

59. A method for treating or preventing a parvovirus infection in a subject, the method comprising administering to the subject an effective amount of the immunogenic composition of claim 42, and generating an immune response in said subject, wherein the immune response prevents or treats a parvovirus infection.
